(19) United States
(12) Patent Application Publication
(10) Pub. No.: US 2008/0178389 A1
(43) Pub. Date: Jul. 31, 2008

(54) BACKBOARD PAD

(76) Inventor: Tanna Sylvia Bender, Snowflake, AZ (US)

Correspondence Address: MICHAEL RIES, 318 PARKER PLACE, OSWEGO, IL, 60543, US

(21) Appl. No.: 12/022,155

(22) Filed: Jan. 30, 2008

Related U.S. Application Data
(60) Provisional application No. 60/898,036, filed on Jan. 30, 2007.

(51) Int. Cl. A47B 1/00; A61F 5/00

(52) U.S. Cl. 5/626; 128/870; 5/625

(57) ABSTRACT

The present invention is a backboard pad constructed of medium density foam. The pad shall provide protection against pressure ulcers caused by extended time on a long spine board after a traumatic accident where spinal injury is suspected. Use of the pad shall also reduce a patients stress whereby decreasing the amount of time needed for recovery.

This application claims priority to U.S. Provisional Application 60/898,036 filed Jan. 30, 2007, Inventor Tanna Bender, confirmation number is 9549, the entire disclosure of which is incorporated by reference.

### TECHNICAL FIELD & BACKGROUND

The present invention generally relates to backboard pads. More specifically, the present invention is a pad that will incorporate the use of medium density foam capable of conforming to each individual patient utilized by emergency services.

The present invention is a pad that will incorporate the use of medium density foam capable of conforming to each individual patient and reanimating it's original form between patients with a durable and washable, and sanitize able cover to be attached to a long spine immobilization board prior to use.

It is mandatory that any person involved in an accident which the patient is left with neck or back pain or simply that the possibility of spinal injury is implied by mechanism, this patient must be placed on a long spine board to maintain inline stabilization reducing risk of further injury.

Currently used long spine boards though very effective in immobilization can in a relatively short amount of time cause pressure ulcers in areas of constant contact. i.e. Posterior region of head shoulders and hips. In addition, these boards are extremely uncomfortable for the patient sometimes increasing pain which can in turn increase stress and blood pressure, neither of which are conducive to a patient's recovery from an already traumatic experience.

Currently, once a patient has been placed on a long spine board for extrication and moved to the ambulance it is sometimes necessary for additional padding to be added using towels or other such padding materials. Unfortunately this will require some movement of the patient to be successful. Any movement regardless of how minimal is not optimal where spinal injuries may be present. Upon arrival at the hospital it is the doctor's goal to remove the patient as quickly as possible from the backboard to prevent aforementioned pressure ulcers as well as reduce pain and stress induced by the discomfort of the board. The doctor may choose, due to time constraints to manually assess the patients C-Spine and remove the patient from a long spine board only to find after the patient has been sent to X-ray that there is indeed a spinal injury at which point the patient must once again be placed on a board requiring more movement of the spine.

With the present pad, the backboard is prepared to cushion and comfort the patient as well as immobilize the patient's spinal column. The benefits will begin immediately by already being in place on the backboard when the patient is extricated onto it. There will be no need for further movement to provide padding as it will already be in place. In turn, the patient will be provided some comfort as well as secure spinal immobilization. Thus, increasing the comfort level the patient's stress level will be lowered allowing for an easier recovery. No longer will the doctor in an overcrowded Emergency room feel hurried to remove a patient for fear of pressure ulcers allowing for more thorough examination prior to movement.

The present invention would be made of a material of medium density foam capable of conforming to each individual patient and reanimating and would not impede necessary X-rays. The pad will measure 72 inches in length and 16 inches wide, the same dimensions of a standard backboard. The pad would be made in different thicknesses to fit different ages and sizes of people. The Pediatric, fitting patients at a weight of 100 pounds or less would be made 1 and ¼ inches thick with a ¾ inch indentation under the head to allow for the pediatrics large occipital bone. The standard adult size would measure 2 and ½ inches in thickness with no indentation under the head.

The XL pad would accommodate the 300 plus pound patient and would be 3 and ½ inches in thickness.

Some of the benefits of the present invention include:

Will facilitate safe and secure transportation of emergency patients.
Will reduce a patient's stress level by providing form fitting comfort as well as inline spinal immobilization.
Sturdy, durable and easy to use.
Washable, waterproof, durable cover allows for sanitizing between patients.
Demographics for such a product would be worldwide being used for all emergency patients both military and civilian.

### BRIEF DESCRIPTION OF THE DRAWINGS

The present invention will be described by way of exemplary embodiments, but not limitations, illustrated in the accompanying drawings in which like references denote similar elements, and in which:

FIG. 1a illustrates a drawing of a backboard pad, in accordance with one embodiment of the present invention; and

FIG. 1b illustrates a drawing of a backboard pad, in accordance with one embodiment of the present invention;

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention, however, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

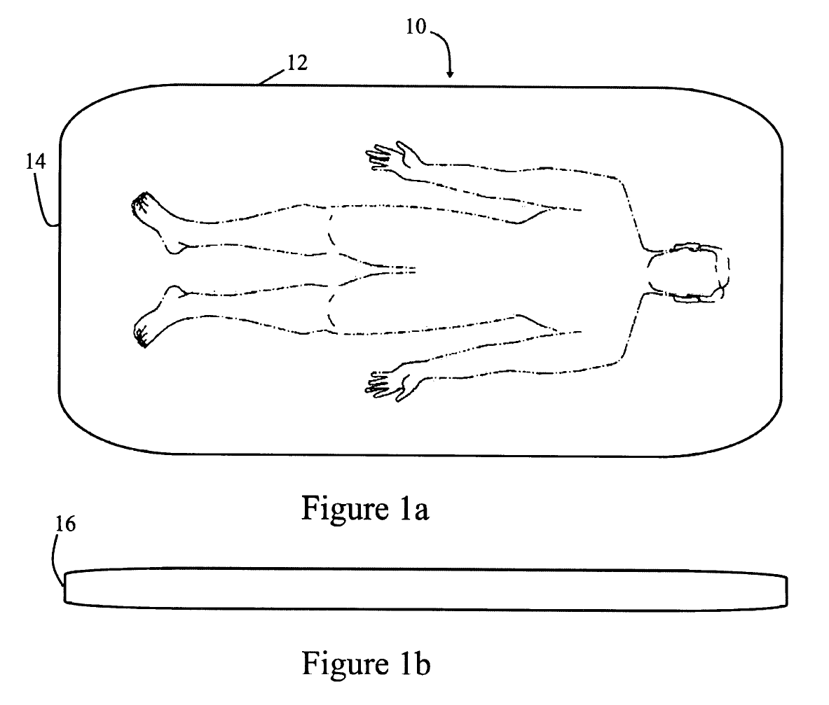

Referring now to FIG. 1a, as in one embodiment illustrated is a top view of a backboard pad 10. Shown is a patient height length 12 that may be seventy two inches. A patient width 14 is shown that may be sixteen inches. In FIG. 1b as in one embodiment a side view shows a pad thickness 16 that may be two or two and one half inches thick.

The backboard pad may be made of medium density memory cell foam and covered with a durable, washable easily sanitized cover. The pad may measure 72 inches in length, 16 inches in width and 2 and ½ inches thick. The backboard pad will adhere to long spine boards used to immobilize people who may possibly be suffering from spinal injuries resulting from auto accidents, rough contact sports, or being struck in the head with strong force. Two strips of heavy duty hook tape approximately 2 inches in width and 72 inches in length will be adhered to each backboard with the corresponding strip of loop tape sewn to the backboard pad.
The backboard pad will be to the benefit of any person who as a result of an accident is placed on a long spine board for immobilization to stabilize and prevent any further spinal injury. The pad will adhere to a long spine board via hook and loop straps prior to needing backboard. Currently when a person is being extricated from a vehicle after an accident they are placed on a long spine board then moved to the ambulance.
Many times once in the ambulance voids under the patient will need to be filled with towels to provide comfort which often requires minimal movement of the patient. In having the backboard pad in place prior to extrication there will be no need for any movement of patient for padding of voids. The backboard pad will automatically fill the voids and seriously reduce chances of pressure ulcers allowing emergency room doctors to take their time and be more thorough before removing the patient from the long spine board with the backboard pad attached. The increased comfort resulting from the pad will also reduce a patient's anxiety and stress levels improving recovery.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

What is claimed is:

1. A backboard pad comprising
a patient height length;
patient width perpendicular to the patent height length; and
a pad thickness perpendicular to a plane crested by the connection of the patient height length and patient width; the backboard pad is made of medium density foam capable of conforming to each individual patient and reanimating, the backboard pad will adhere to a long spine board.

2. The backboard pad of claim 1 wherein the backboard pad is covered with a durable, washable easily sanitized cover.

3. The backboard pad of claim 1 wherein the patient height length is 72 inches the patient width is 16 inches and the pad thickness is 2 and ½ inches.

4. The backboard pad of claim 1 wherein the backboard pad will adhere to a long spine board via hook and loop straps prior to needing the backboard pad.
